(19) United States
(12) Patent Application Publication
(10) Pub. No.: US 2022/0192467 A1
(43) Pub. Date: Jun. 23, 2022

(54) Method and Apparatus for Extending Battery Life of Capsule Endoscope

(71) Applicant: CAPSOVISION, Inc., Saratoga, CA (US)

(72) Inventors: Kang-Huai Wang, Saratoga, CA (US); Mark Hadley, Los Altos, CA (US); Chang-Wei Lin, Mountain View, CA (US); Chung-Ta Lee, Sunnyvale, CA (US)

(21) Appl. No.: 17/128,142

(22) Filed: Dec. 20, 2020

(51) Int. Cl. A61B 1/00; A61B 1/04; A61B 1/045; A61B 1/06

(52) U.S. Cl. CPC A61B 1/00036; A61B 1/041; A61B 1/00016; A61B 1/0684; A61B 1/0002; A61B 1/045

(57) ABSTRACT

Method for extending battery life and a capsule endoscope using the method are disclosed. According to this method, sub-tasks associated with capturing one image using the capsule endoscope when the capsule endoscope moves through a human GI (gastrointestinal) tract after being ingested by a human subject are identified. The capsule endoscope is capable of capturing an image sequence at a first frame period, and at least two sub-tasks are performed with partial or full overlap. One or more images are captured at a second frame period by performing sub-task spreading, where the sub-task spreading spreads the sub-tasks over time to reduce or avoid the partial or full overlap for said at least two sub-tasks so as to reduce a sub-task peak current or peak current duration. The second frame period is longer than the first frame period.

### FIELD OF THE INVENTION

The present invention relates to battery-powered devices, including capsule cameras for imaging the gastrointestinal (GI) tract. In particular, the present invention discloses methods to extend battery life so that the capsule cameras operate at low peak current at low frame rate while capable of high frame rate when needed.

### BACKGROUND AND RELATED ART

Devices for imaging body cavities or passages in vivo are known in the art and include endoscopes and autonomous encapsulated cameras. Endoscopes are flexible or rigid tubes that pass into the body through an orifice or surgical opening, typically into the esophagus via the mouth or into the colon via the rectum. An image is formed at the distal end using a lens and transmitted to the proximal end, outside the body, either by a lens-relay system or by a coherent fiber-optic bundle. A conceptually similar instrument might record an image electronically at the distal end, for example using a CCD or CMOS array, and transfer the image data as an electrical signal to the proximal end through a cable. Endoscopes allow a physician control over the field of view and are well-accepted diagnostic tools. However, they do have a number of limitations, present risks to the patient, are invasive and uncomfortable for the patient, and their cost restricts their application as routine health-screening tools.

Because of the difficulty traversing a convoluted passage, endoscopes cannot easily reach the majority of the small intestine and special techniques and precautions, that add cost, are required to reach the entirety of the colon. Endoscopic risks include the possible perforation of the bodily organs traversed and complications arising from anesthesia. Moreover, a trade-off must be made between patient pain during the procedure and the health risks and post-procedural down time associated with anesthesia.

An alternative in vivo image sensor that addresses many of these problems is the capsule endoscope. A camera is housed in a swallowable capsule, along with a radio transmitter for transmitting data, primarily comprising images recorded by the digital camera, to a base-station receiver or transceiver and data recorder outside the body. The capsule may also include a radio receiver for receiving instructions or other data from a base-station transmitter. Instead of radio-frequency transmission, lower-frequency electromagnetic signals may be used. Power may be supplied inductively from an external inductor to an internal inductor within the capsule or from a battery within the capsule.

An autonomous capsule camera system with on-board data storage was disclosed in the U.S. Pat. No. 7,983,458, entitled “In Vivo Autonomous Camera with On-Board Data Storage or Digital Wireless Transmission in Regulatory Approved Band,” granted on Jul. 19, 2011. This patent describes a capsule system using on-board storage such as semiconductor nonvolatile archival memory to store captured images. After the capsule passes from the body, it is retrieved. Capsule housing is opened and the images stored are transferred to a computer workstation for storage and analysis. For capsule images either received through wireless transmission or retrieved from on-board storage, the images will have to be displayed and examined by diagnostician to identify potential anomalies.

FIG. 1 illustrates an exemplary capsule system with on-board storage, where the capsule camera is in the human gastrointestinal (GI) tract 100. The capsule system 110 includes illuminating system 12 and a camera that includes optical system 14 and image sensor 16. A semiconductor nonvolatile archival memory 20 may be provided to allow the images to be stored and later retrieved at a docking station outside the body, after the capsule is recovered. System 110 includes battery power supply 24 and an output port 26. Capsule system 110 may be propelled through the GI tract by peristalsis.

Illuminating system 12 may be implemented by LEDs. In FIG. 1, the LEDs are located adjacent to the camera's aperture, although other configurations are possible. The light source may also be provided, for example, behind the aperture. Other light sources, such as laser diodes, may also be used. Alternatively, white light sources or a combination of two or more narrow-wavelength-band sources may also be used. White LEDs are available that may include a blue LED or a violet LED, along with phosphorescent materials that are excited by the LED light to emit light at longer wavelengths. The portion of capsule housing 10 that allows light to pass through may be made from bio-compatible glass or polymer.

Optical system 14, which may include multiple refractive, diffractive, or reflective lens elements, provides an image of the lumen walls on image sensor 16. Image sensor 16 may be provided by charged-coupled devices (CCD) or complementary metal-oxide-semiconductor (CMOS) type devices that convert the received light intensities into corresponding electrical signals. Image sensor 16 may have a monochromatic response or include a color filter array such that a color image may be captured (e.g. using the RGB or CYM representations). The analog signals from image sensor 16 are preferably converted into digital form to allow processing in digital form. Such conversion may be accomplished using an analog-to-digital (A/D) converter, which may be provided inside the sensor (as in the current case), or in another portion inside capsule housing 10. The A/D unit may be provided between image sensor 16 and the rest of the system. LEDs in illuminating system 12 are synchronized with the operations of image sensor 16. Processing module 22 may be used to provide processing required for the system such as image processing and video compression. The processing module may also provide needed system control such as to control the LEDs during image capture operation. The processing module may also be responsible for other functions such as managing image capture and coordinating image retrieval.

After the capsule camera traveled through the GI tract and exits from the body, the capsule camera is retrieved and the images stored in the archival memory are read out through the output port. The received images are usually transferred to a base station for processing and for a diagnostician to examine. The accuracy as well as efficiency of diagnostics is most important. A diagnostician is expected to examine all images and correctly identify all anomalies. Furthermore, it is desirable to gather location information of the anomalies, which is useful for possible operations or treatment of the anomalies. While various location detection devices could be embedded or attached to the capsule device, it is desirable to develop methods for determining the travelled distance based on images captured.

For capsule cameras, the entire GI examination process is powered by the internal battery. It may take more than 20 hours from the moment that the capsule camera is swallowed to the moment it is excreted from the human body. During the examination process, thousands of images will be captured, processed, and then either be stored on-board or wirelessly transmitted to an external receiver. Due to the small capsule size, the capsule device can only afford very limited power capacity. Therefore, it is very critical to utilize the battery capacity wisely in order to optimize the battery capacity usage.

For capsule endoscope application, cell batteries or button batteries are often used. There are different types of materials (i.e., chemical compositions) used for the batteries, such as alkaline, lithium and silver oxide. For each battery, there is a nominal capacity specified and the capacity is often quoted in milliamp hour (mAh). For example, an SR927 battery may have a capacity of 60 mAh at 1.2V cutoff. The battery has a nominal voltage of 1.55V and is intended for devices operated at about 1.5V. While the battery is designed to maintain relatively constant output voltage, however, the output voltage drops substantially when the battery becomes depleted. Therefore, the nominal capacity of the battery may not be fully utilized as specified. One important factor influencing the usable capacity is the battery load. In general, a load that draws a higher drain current will have lower usable capacity due to various reasons such as internal resistance, polarization effect and/or undesirable chemical reactions inside the battery.

FIG. 2 illustrates an example of battery output voltage vs. discharge capacity for two different drain currents. The drawing is intended to illustrate the nature of usable capacity or discharge capacity at different drain currents (I_{A} and I_{B}). In FIG. 2, curves 210 and 220 correspond to the output voltage vs. discharge capacity at drain currents I_{A} and I_{B} respectively, where I_{A}>I_{B}. When the output voltage drops below a certain level (cutoff voltage 230), the battery may not be able to provide the power for the connected device to operate properly. As shown in FIG. 2, at a higher drain current I_{A}, the battery will deliver a lower usable capacity Cap_{A}. On the other hand, at a lower drain current I_{B}, the battery will deliver a higher usable capacity Cap_{B}.

The present invention discloses methods to extend battery life for a device powered by batteries. While the capsule endoscope is illustrated as a such device, the present invention can be used in other electronic devices powered by batteries.

### BRIEF SUMMARY OF THE INVENTION

A method and a capsule endoscope incorporating the method are disclosed. According to the method, sub-tasks associated with capturing one image using the capsule endoscope when the capsule endoscope moves through a human GI (gastrointestinal) tract after being ingested by a human subject is identified, where the capsule endoscope is capable of capturing an image sequence at a first frame period, and at least two sub-tasks are performed with partial or full overlap. One or more images are captured by performing sub-task spreading, wherein said sub-task spreading spreads the sub-tasks over time to reduce or avoid the partial or full overlap for said at least two sub-tasks so as to reduce a peak current or peak current duration, and wherein the second frame period is longer than the first frame period.

In one embodiment, the sub-tasks comprise image sensing, image processing, and pre-charging LED light source. If the capsule endoscope includes an archive memory, the sub-tasks further comprise image write to the archive memory. If the capsule endoscope includes an internal wireless transmitter, the sub-tasks further comprise image transmission to an external wireless receiver using the internal wireless transmitter.

In one embodiment, the sub-tasks are spread so that an overlap between two sub-tasks is reduced.

In one embodiment, the sub-tasks are spread so that a duration for one sub-task is extended.

In one embodiment, the sub-tasks are spread so that a duration for one highest-current sub-task is extended.

According to another method, a set of sub-tasks associated with capturing one image using the capsule endoscope when the capsule endoscope moves through a human GI (gastrointestinal) tract after being ingested by a human subject are identified, where the capsule endoscope is capable of capturing one image frame according to the set of sub-tasks at a first frame period, and the set of sub-tasks at the first frame period results in a sub-task peak current. The set of sub-tasks are executed to capture one or more images at a second frame period by lengthening a duration for one sub-task to lower the sub-task peak current or by lengthening a gap between two sub-tasks, and wherein the second frame period is longer than the first frame period.

In one embodiment, said one sub-task corresponds to a target sub-task having a highest current among the set of sub-tasks.

### BRIEF DESCRIPTION OF THE DRAWINGS

FIG. 1 illustrates an exemplary capsule system with on-board storage, where the capsule system includes illuminating system and a camera that includes optical system and image sensor.

FIG. 2 illustrates an example of battery output voltage vs. discharge capacity for two different drain currents.

FIG. 3A illustrates an example of current consumption profiles for the sub-tasks, where the drawing shows the capsule endoscope is operated at near full duty cycle, and the cycle of a next task starts shortly after a current task is completed.

FIG. 3B illustrates an example of current consumption profiles at a low duty cycle, which corresponds to two task cycles per time unit T_{U}.

FIG. 3C illustrates an example of total current consumption profile for the sub-tasks in FIG. 3A and FIG. 3B.

FIG. 4A illustrates an example of spreading the sub-tasks to reduce or avoid the overlap between at least two sub-tasks according to one embodiment of the present invention.

FIG. 4B illustrates an example of reduced peak current for the sub-tasks incorporating sub-task spreading as shown in FIG. 4A.

FIG. 4C illustrates an example of separating sub-tasks further apart compared to the sub-tasks in FIG. 4A.

FIG. 4D illustrates an example of sub-task peak current profiles for the sub-tasks in FIG. 4C.

FIG. 5 illustrates an exemplary flowchart for a capsule endoscope incorporating sub-task spreading according to an embodiment of the present invention.

FIG. 6 illustrates another exemplary flowchart for a capsule endoscope incorporating sub-task spreading according to an embodiment of the present invention.

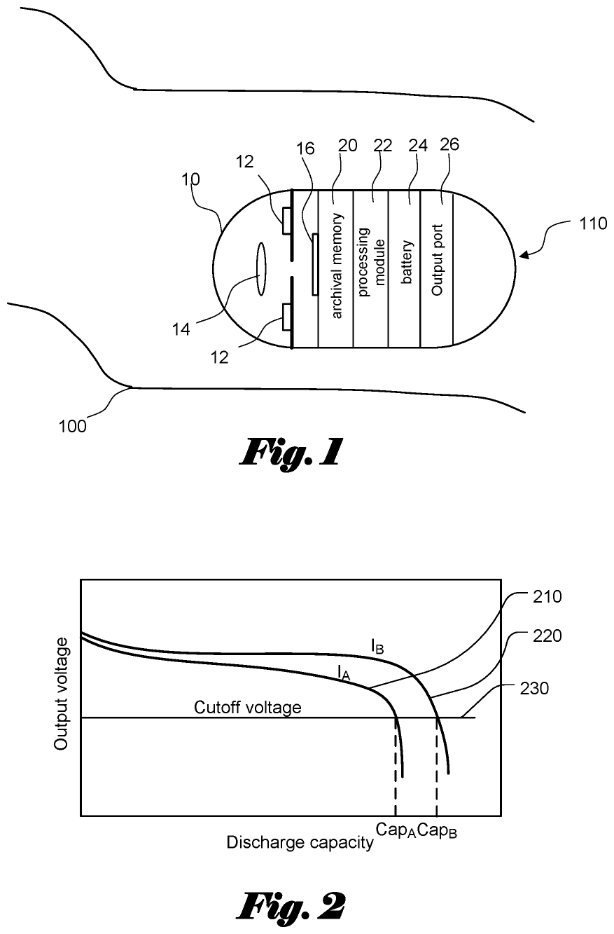

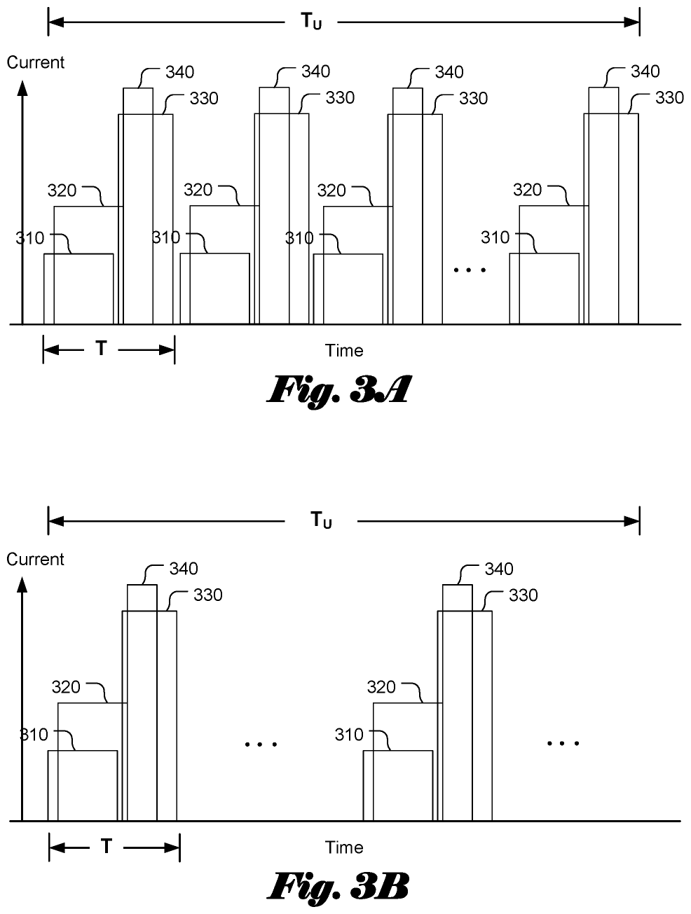

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely representative of selected embodiments of the invention. References throughout this specification to “one embodiment,” “an embodiment,” or similar language mean that a particular feature, structure, or characteristic described in connection with the embodiment may be included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention. The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

Endoscopes are normally inserted into the human body through a natural opening such as the mouth or anus. Therefore, endoscopes are preferred to be small sizes so as to be minimally invasive. As mentioned before, endoscopes can be used for diagnosis of human gastrointestinal (GI) tract. The captured image sequence can be viewed to identify any possible anomaly. The capsule endoscope may take more than 10 hours to travel through the human GI tract before it is excreted. During the course of travelling through the human GI tract, the capsule endoscope may have to capture tens of thousands images of the GI tract, process the images, and either store images on-board or transmit the images to external receiver. The capsule endoscope has to execute all the related tasks based on the power from one or more tiny batteries inside the capsule housing. Accordingly, the power is a very precious resource to operate the capsule endoscope. Therefore, it is desirable to squeeze out as much power from the batteries as possible. This is also true for many battery-powered electronic devices.

The capsule endoscope moves slowly in the GI tract. Due to the slow movement, the capsule endoscope usually captures images at a very slow frame rate, such as 2-5 frames per second. The current image sensor technology and the circuit technology are capable of capturing and processing images at a much higher frame rate, such as 10 frames per second. Accordingly, the capsule endoscope is usually operated at a relatively low duty cycle.

Let's assume that one task will take duration T to complete, which results in 1/T repetitions per second for the task. For example, T corresponds to 0.1 second, which results in a maximum throughput rate of 10 repetitions of the task per second. In the case of a capsule endoscope with on-board storage, each task comprises capturing the image, processing the image and storing the image on-board. In this example, the capsule endoscope is capable of handling 10 frames per second. If we take 3 frames per second, then the capsule endoscope is idle 70% of the time. In other words, the “duty cycle” is 30%.

In a capsule endoscope, each image capture (either recorded or transmitted) can be considered as a task. The sub-activities of image capture task in terms of current consumption may comprise image sensing by the image sensor, image processing by the processor, flash memory writing, and lighting energy (e.g., LED light) pre-charge for the subsequent image sensing, with some of them overlapped in time. These sub-tasks are just an example to illustrates the incorporating the present invention in a capsule endoscope. For other battery powered devices, different sub-tasks may be identified for the target task. These sub-tasks are often performed with some overlap. For example, the image sensing sub-task will output image data on a line-by-line basis. Image processing, such as de-mosaicking and image compression may start upon sufficient image data (e.g., 8 lines) collected. The flash memory write can start after sufficient image data are processed. On the other hand, the LED capacitor can be pre-charged after the current image is sensed so that the LED light can be ready to trig for the next frame when needed.

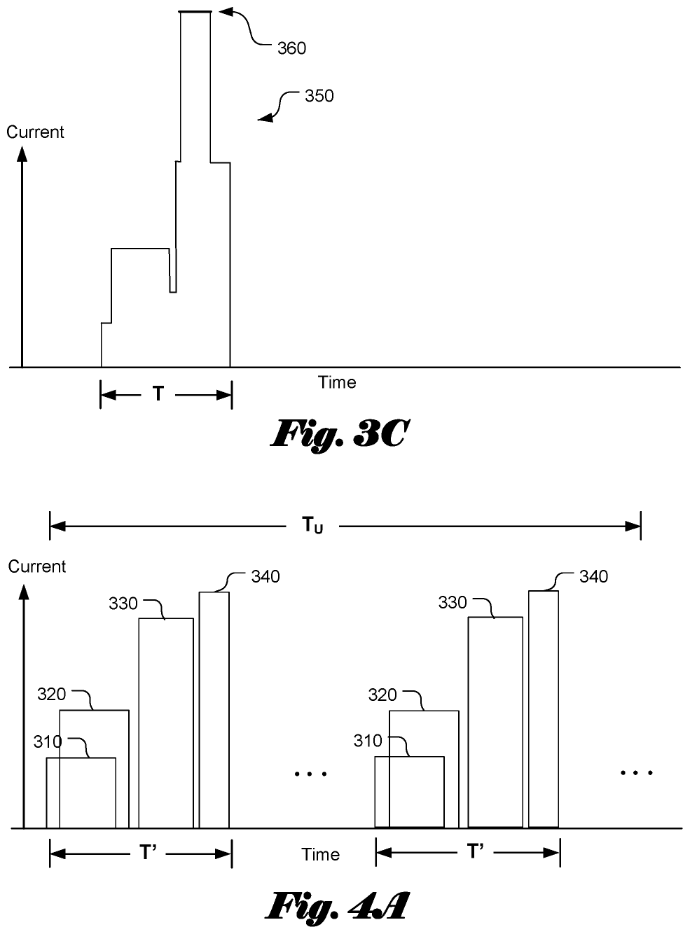

FIG. 3A illustrates an example of current consumption profiles for the sub-tasks, where the drawing shows the capsule endoscope is operated at near full duty cycle, and the cycle of a next task starts shortly after a current task is completed. The duration for each cycle is T and there are T_{U}/T tasks that can be executed during a time unit T_{U}. The time unit can be any specified period, such as 1 second. The higher frame rate sometimes is needed at a certain part of GI tract, such as esophagus, or when fast motion is detected that triggers a high frame rate. As mentioned earlier, the capsule endoscope is often operated at low duty cycles. The example in FIG. 3B corresponds to two task cycles per time unit T_{U}. Therefore, there is a long idle time between two tasks. The major tasks illustrated in the example of FIG. 3A correspond to image sensing 310, image processing 320, flash memory write 330 and LED capacitor pre-charge for the next sensing 340. In FIG. 3A, the current profiles of the sub-activities are shown as box shapes for the illustration purpose. In practice, the current profile may be smooth curves having a rise time and a fall time due to non-zero transistor turn-on time and capacitance exiting in the load circuits between VCC and GND. When two sub-tasks overlap, the total current consumption adds up. For example, the image sensing 310 and image processing 320 have substantial overlap and the total current consumption corresponds to the sum of two individual currents during the overlapped period. Also, the LED capacitor pre-charge for the next sensing sub-task 340 fully overlaps with the flash memory write sub-task 330. Therefore, the total current consumption during the LED capacitor pre-charge time corresponds to the sum of currents for the LED capacitor pre-charge and the flash memory write. The total current consumption profile 350 of the sub-tasks is shown in FIG. 3C. The peak current 360 occurs during the overlapped period of the LED capacitor pre-charge and the flash memory write. The same situation can be applied to cell phones. For cell phones, depending on available bandwidth, the frame rate in video conferencing may be only a fraction of the fastest frame rate that the device is capable of. Similar situation may occur in a dark environment, where the frame rate has to be reduced for the pixels to collect sufficient photons. In the case of recording a video clip in sport event, the required frame rate may be substantially higher than 30 frames per second, say 45 FPS, while a frame rate at 30 FPS or less is used in more frequent daily applications. The sub-tasks can be more spread out at 30 FPS than in 45 FPS without reducing the battery capacity available as in 45 FPS case. In FIG. 3C, the vertical scale is compressed compared to that of FIG. 3A and FIG. 3B in order to properly fit the drawing within the page.

In certain cases, some sub-tasks are interchangeable. For example, in the above case, the writing to flash memory and pre-charging the capacitor to store energy for LED lighting for the next frame are interchangeable, as long as the peak current is reduced by spreading out the sub-tasks. The present invention also covers variations of sub-task spreading, such as reversing the sequence order of the sub-tasks or changing the sequence order if the orders in the sequence are interchangeable.

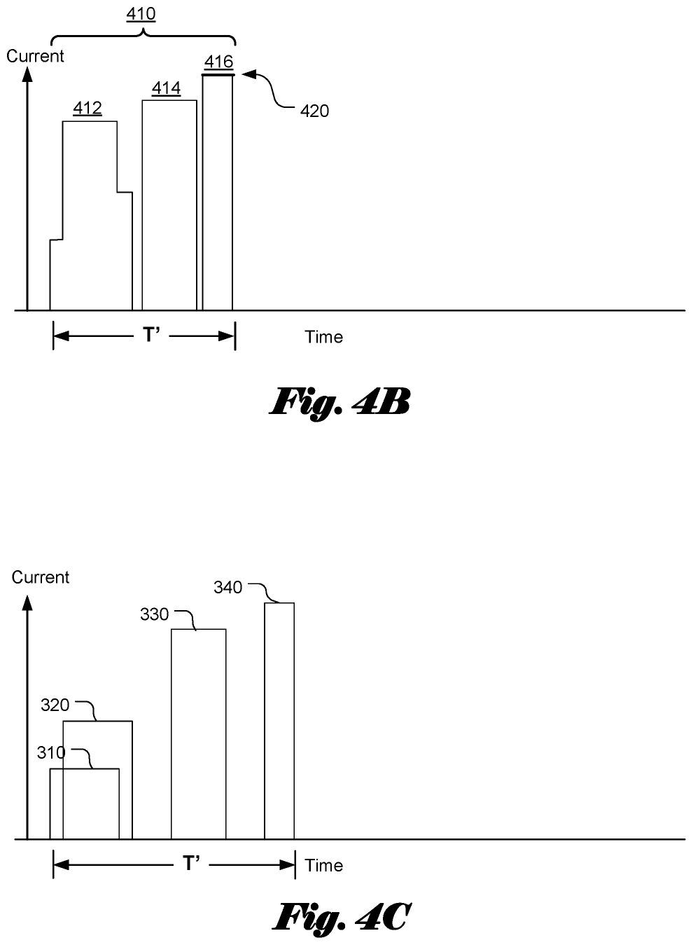

In order to lower the peak current, the present invention disclose a method to spread these sub-tasks into a wider duration to reduce or avoid the overlap among sub-tasks. This would result in a slower frame rate. Nevertheless, since the capsule endoscope is often operated in low duty cycle and the capsule endoscope can afford to operate at a lower frame rate. Therefore, the present invention can reduce the peak current by spreading the current in a wider duration without compromising the intended performance. FIG. 4A illustrates an example of spreading the sub-tasks to avoid overlap between at least two sub-tasks, where said at least two sub-tasks are overlapped when the capsule is operated at a higher frame rate, according to one embodiment of the present invention. In FIG. 4A, the LED capacitor pre-charge for the next sensing sub-task 340 becomes separated from the flash memory write sub-task 330. There is no more overlap between sub-task 330 and sub-task 340. The peak current profile 410 for sub-task peak currents (412, 414 and 416) being spread is shown in FIG. 4B, where the peak current consumption is reduced to the pre-charging current 420. Compared to the peak current in FIG. 3C, the peak current according to the present invention is substantially reduced. Sub-task 330 and sub-task 340 are rather independent and can be inter-exchanged. While the flash memory write sub-task 330 is performed before the LED capacitor pre-charge for the next sensing sub-task 340 in FIG. 4A, the LED capacitor pre-charge for the next sensing sub-task 340 can be performed after the flash memory write sub-task 330.

The recovery time may also play a factor in the capacity available from a battery when the task rate is low. Therefore, it is preferable not only to avoid the overlap between sub-tasks, but also to separate high current sub-tasks to further apart. FIG. 4C illustrates an example of separating sub-tasks further apart compared to the sub-tasks in FIG. 4A. While sub-tasks 330 and 340 are spread to avoid overlap in FIG. 4A, sub-tasks 330 and 340 are further spread so that the gap between sub-task 330 and sub-tasks 310/320 becomes larger, and the gap between sub-task 330 and sub-task 340 also becomes larger in FIG. 4C. The sub-task peak current profiles (432, 434 and 436) are shown in FIG. 4D.

As is known in the field, a battery at a higher drain current will provide less total energy than it will at a lower drain current. Therefore, embodiments of the present invention to spread the sub-tasks to reduce or avoid any overlap between two sub-tasks will allow the capsule endoscope to derive more electrical capacity from the batteries. Beside spreading the sub-tasks to reduce or avoid overlap, individual sub-tasks may also be spread as long as the system has sufficient time to complete the task. For example, the LED may take a longer duration to pre-charge so as to reduce the charging current. In another example, both LED pre-charge and memory write sub-tasks may spread the duration so that both sub-tasks will result in reduced peak current consumption.

For digital part of the functions with lower performance requirements (e.g., lower frame rate), lowering the clock can lower the peak current. While this is one way to lower the peak current, there are other ways to reduce or avoid the parallel processing of sub-tasks when possible, but still meet the performance requirement. This can be realized by one or more control circuits. It may also be flexibly realized by a CPU or a multiple-core system, or a combination of CPU, one or more processors, and one or more circuits. For example, a smartphone may be performing MPEG video compression while performs facial recognition in real time on each image. At a fast frame rate, substantial overlap of these two functions or sub-tasks may be required. However, at a lower frame rate, the overlap between these two functions or sub-tasks may be reduced or avoided.

In yet another embodiment, the sub-task spreading is achieved by performing some selected sub-tasks over longer periods of time. For example, the sub-task spreading can be achieved by slowing down the operating clock so that the duration for a target sub-task is increased by a factor depending on the ratio of the original clock and the slowed-down clock. In this case, the target sub-task is spread so that the consumed current by the target sub-task is reduced over a longer period. Similar to the method of slowing down the clock, if a target sub-task is implemented using one or more processors, CPUs or ASIC, said one or more processors, CPUs or ASIC can be programmed to take linger time to finish the target sub-task. For example, AISC may be used to implement image compression (i.e., part of image processing) and the ASIC can be configured to take twice as long to finish the target sub-task in order to reduce the peak current associated with the target sub-task. In another example, the target sub-task corresponds to the LED pre-charge current, where a capacitor is often used to hold the charges for triggering the LED light. In this case, we could lower the pre-charge current by charging it over a longer period of time. The pre-charge current may be controlled by a CPU via some current control circuits.

In some cases, the system time may be constrained so that sub-tasks cannot be fully spread to avoid overlap between two sub-tasks. However, as long as the overlap duration is reduced, the duration of high peak current consumption is reduced and it helps to derive higher battery capacity.

In the above discussion, the capsule endoscope is used to illustrate the method of extending battery life by spreading the sub-tasks to lower the peak current consumption. However, the present invention is not limited to the capsule endoscope. Any battery powered device, such as IoT (Internet of Thing) devices, wearable devices or smart phones can be benefitted from the present invention by adopting sub-task spreading.

The method mentioned above can be implemented using various programmable devices such as micro-controller, central processing unit (CPU), field programmable gate array (FPGA), digital signal processor (DSP), ASIC (Application Specific Integrated Circuit) or any programmable processor or circuitry.

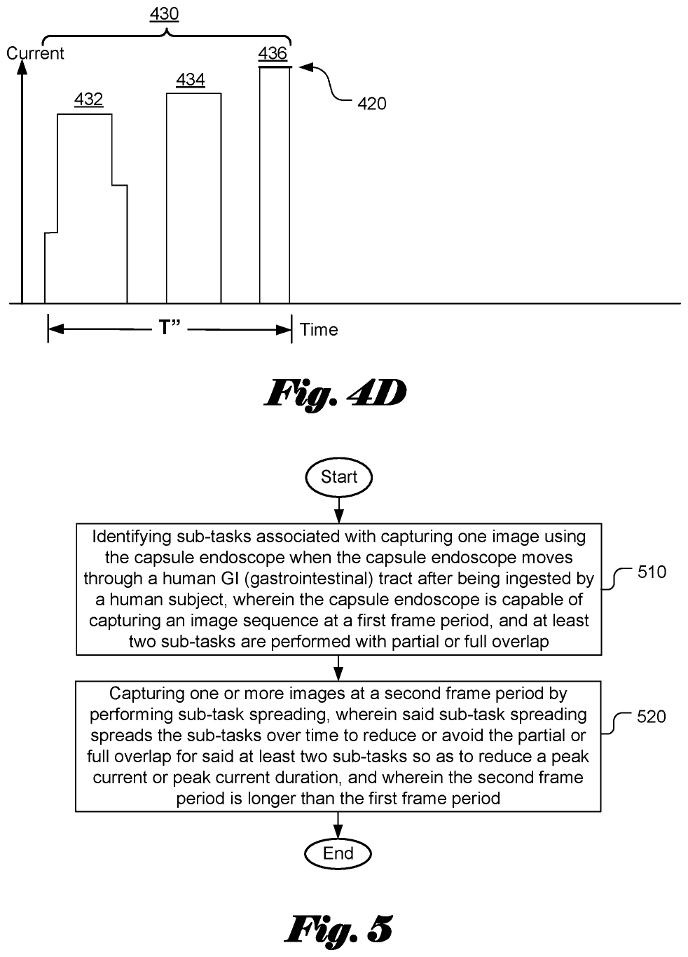

FIG. 5 illustrates an exemplary flowchart for a capsule endoscope incorporating sub-task spreading according to an embodiment of the present invention. According to this method, sub-tasks associated with capturing one image using the capsule endoscope when the capsule endoscope moves through a human GI (gastrointestinal) tract after being ingested by a human subject are identified in step 510, wherein the capsule endoscope is capable of capturing an image sequence at a first frame rate, and at least two sub-tasks are performed with partial or full overlap. One or more images are captured a second frame rate by performing sub-task spreading in step 520, wherein said sub-task spreading spreads the sub-tasks over time to reduce or avoid the partial or full overlap for said at least two sub-tasks so as to reduce a peak current or peak current duration, and wherein the second frame rate is smaller than the first frame rate.

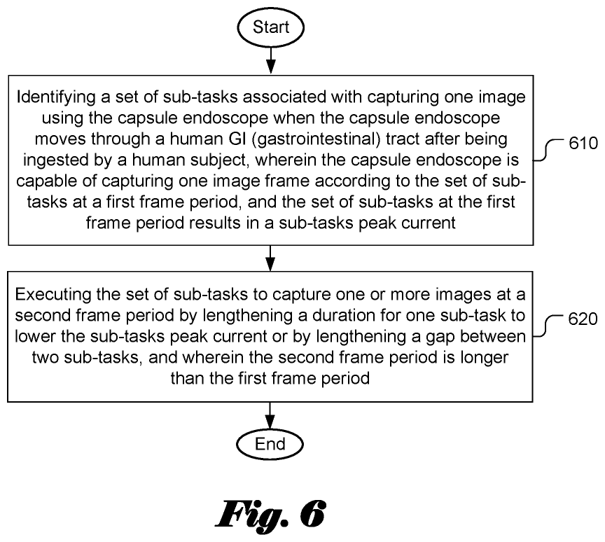

FIG. 6 illustrates another exemplary flowchart for a capsule endoscope incorporating sub-task spreading according to an embodiment of the present invention. According to this method, a set sub-tasks associated with capturing one image using the capsule endoscope when the capsule endoscope moves through a human GI (gastrointestinal) tract after being ingested by a human subject are identified in step 610, wherein the capsule endoscope is capable of capturing one image frame according to the set of sub-tasks at a first frame period, and the set of sub-tasks at the first frame period results in a sub-task peak current. The set of sub-tasks is executed to capture one or more images at a second frame period by lengthening a duration for one sub-task to lower the sub-task peak current or by lengthening a gap between two sub-tasks in step 620, and wherein the second frame period is longer than the first frame period.

The above description is presented to enable a person of ordinary skill in the art to practice the present invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In the above detailed description, various specific details are illustrated in order to provide a thorough understanding of the present invention. Nevertheless, it will be understood by those skilled in the art that the present invention may be practiced.

The invention may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method of extending battery life for a capsule endoscope powered by a battery, the method comprising:
identifying sub-tasks associated with capturing one image using the capsule endoscope when the capsule endoscope moves through a human GI (gastrointestinal) tract after being ingested by a human subject, wherein the capsule endoscope is capable of capturing an image sequence at a first frame period, and at least two sub-tasks are performed with partial or full overlap; and
capturing one or more images at a second frame period by performing sub-task spreading, wherein said sub-task spreading spreads the sub-tasks over time to reduce or avoid the partial or full overlap for said at least two sub-tasks so as to reduce a peak current or peak current duration, and wherein the second frame period is longer than the first frame period.

2. The method of claim 1, wherein the sub-tasks comprise image sensing, image processing, and pre-charging LED light source.

3. The method of claim 2, wherein the sub-tasks further comprise image write to an archive memory.

4. The method of claim 2, wherein the sub-tasks further comprise image transmission to an external wireless receiver.

5. The method of claim 1, wherein the sub-tasks are spread so that an overlap between two sub-tasks is reduced.

6. The method of claim 1, wherein the sub-tasks are spread so that a duration for one sub-task is extended.

7. The method of claim 1, wherein the sub-tasks are spread so that a duration for one highest-current sub-task is extended.

8. A capsule endoscope, comprising:
a pixel array being responsive to light energy received by the pixel array;
an LED light source to illuminate a scene for the pixel array;
one or more circuits coupled to the pixel array and the LED light source; and
a battery to supply electrical power to the pixel array, the LED light source and said one or more circuits; and
a housing adapted to be swallowed, wherein the battery, the pixel array, the LED light source and said one or more circuits are enclosed in the housing;
wherein said one or more circuits, the pixel array and the LED light source are capable of capturing an image sequence at a first frame period, and wherein a task associated with capturing one image comprise a plurality of sub-tasks and at least two sub-tasks are performed with partial or full overlap; and
wherein said one or more circuits, the pixel array and the LED light source are configured to:
capture one or more images at a second frame period by performing sub-task spreading, wherein said sub-task spreading spreads the sub-tasks over time to reduce or avoid the partial or full overlap for said at least two sub-tasks so as to reduce a peak current or peak current duration, and wherein the second frame period is longer than the first frame period.

9. The capsule endoscope of claim 8, wherein the sub-tasks comprise image sensing, image processing, and pre-charging LED light source.

10. The capsule endoscope of claim 9, further comprises an archive memory, wherein the sub-tasks further comprise image write to the archive memory.

11. The capsule endoscope of claim 9, further comprises an internal wireless transmitter, wherein the sub-tasks further comprise image transmission to an external wireless receiver using the internal wireless transmitter.

12. The capsule endoscope of claim 8, wherein the sub-tasks are spread so that an overlap between two sub-tasks is reduced.

13. The capsule endoscope of claim 8, wherein the sub-tasks are spread so that a duration for one sub-task is extended.

14. The capsule endoscope of claim 8, wherein the sub-tasks are spread so that a duration for one highest-current sub-task is extended.

15. A method of extending battery life for a capsule endoscope powered by a battery, the method comprising:
identifying a set of sub-tasks associated with capturing one image using the capsule endoscope when the capsule endoscope moves through a human GI (gastrointestinal) tract after being ingested by a human subject, wherein the capsule endoscope is capable of capturing one image frame according to the set of sub-tasks at a first frame period, and the set of sub-tasks at the first frame period results in a sub-task peak current; and
executing the set of sub-tasks to capture one or more images at a second frame period by lengthening a duration for one sub-task to lower the sub-task peak current or by lengthening a gap between two sub-tasks, and wherein the second frame period is longer than the first frame period.

16. The method of claim 15, wherein said one sub-task corresponds to a target sub-task having a highest current among the set of sub-tasks.

17. A capsule endoscope, comprising:
a pixel array being responsive to light energy received by the pixel array;
an LED light source to illuminate a scene for the pixel array;
one or more circuits coupled to the pixel array and the LED light source; and
a battery to supply electrical power to the pixel array, the LED light source and said one or more circuits; and
a housing adapted to be swallowed, wherein the battery, the pixel array, the LED light source and said one or more circuits are enclosed in the housing;
wherein said one or more circuits, the pixel array and the LED light source are capable of capturing one image frame according to a set of sub-tasks at a first frame period and the set of sub-tasks at the first frame period results in a sub-task peak current; and
wherein said one or more circuits, the pixel array and the LED light source are configured to execute the set of sub-tasks to capture one or more images at a second frame period by lengthening a duration for one sub-task to lower the sub-task peak current or by lengthening a gap between two sub-tasks, and wherein the second frame period is longer than the first frame period.

18. The capsule endoscope of claim 17, wherein said one sub-task corresponds to a target sub-task having a highest current among the set of sub-tasks.
